(19) United States
(12) Patent Application Publication
(10) Pub. No.: US 2022/0272968 A1
(43) Pub. Date: Sep. 1, 2022

(54) Non-Woven Antibacterial Hand Glove

(71) Applicant: Joshua Postell, Atlanta, GA (US)

(72) Inventor: Joshua Postell, Atlanta, GA (US)

(21) Appl. No.: 16/993,667

(22) Filed: Aug. 14, 2020

(51) Int. Cl. A01N 25/34; A61L 2/00; A01N 25/10; A01N 25/02; A01N 31/02; A01P 1/00; A41D 31/30

(52) U.S. Cl. CPC A01N 25/34; A61L 2/0088; A01N 25/10; A01N 25/02; A01N 31/02; A01P 1/00; A41D 31/30; A61L 2101/34

(57) ABSTRACT

Methods and devices for rendering hand gloves antibacterial are disclosed. A hand glove comprising a non-woven, porous, and/or hydrophobic sheet can be impregnated with a sanitizing-based solution on an exterior surface. The sanitizing-based solution, which can be a porous alcohol or a non-alcohol based solution that contains functional ingredients can be used to kill germs and bacteria during initial contact with surfaces. After impregnation with the sanitizing-based solution, the hand glove can provide functionality that allows the sanitizing-based solution to uniformly adhere to the hand glove such that a gloved hand can touch and clean surfaces while keeping hands protected and safeguarded.

### FIELD OF INVENTION

The present invention relates to an antibacterial hand glove. The antibacterial hand glove can be non-woven, porous, and/or hydrophobic sheet. The sheet can be impregnated with an alcohol or non-alcohol-based sanitizer solution that includes polymeric beads, which are characterized in providing controlled release of a functional ingredient.

### BACKGROUND

A variety of treated cloths which are adapted for skin care are commercially available. Such products typically comprise paper or non-woven hydrophobic or permeable fabric sheets which are wetted with an aqueous sanitizing solution of alcohol based or non-alcohol solution that is soluble or water dispersible ingredients. The benefit presented by these ingredients is that they are typically water insoluble or hydrophobic. However, emulsions tend to be unstable. Further, the chemicals that emulsify hydrophobic materials also tend to keep such components from uniformly adhering to the human hand.

Therefore, it would be advantageous to provide hydrophobic functional material as well as option permeable material in the form of an antibacterial hand glove that allows disbursement of the sanitizing solution in a stable form.

It is a further object of the present invention to controllably release such functional ingredients when applied to the human skin or environmental surfaces.

Other objects advantages and novel features of the present invention will be apparent to those skilled in the art from the following description and appended claims.

### SUMMARY

Examples of the present disclosure comprise methods and devices for rendering hand gloves antibacterial. The device can include a hand glove comprising a non-woven, porous, and hydrophobic sheet that can be impregnated with a sanitizing solution at an exterior surface of the sheet. Once impregnated, the hand glove can be rendered antibacterial while preventing the sanitizing, solution from adhering to the hand when worn.

Further features of the disclosed design, and the advantages offered thereby, are explained in greater detail hereinafter with reference to specific examples illustrated in the accompanying drawings, wherein like elements, are indicated be like reference designators.

### BRIEF DESCRIPTION OF THE DRAWINGS

Reference will now be made to the accompanying drawings, which are not necessarily drawn to scale, are incorporated into, and constitute a portion of, this disclosure, illustrate various implementations and aspects of the disclosed technology and, together with the description, serve to explain the principles of the disclosed technology. In the drawings:

FIG. 1 is a front view of an example of an antimicrobial hand glove, in accordance with some examples of the present disclosure; and

FIG. 2 is a perspective view of an example of an antimicrobial hand glove, in accordance with some examples of the present disclosure.

### DETAILED DESCRIPTION

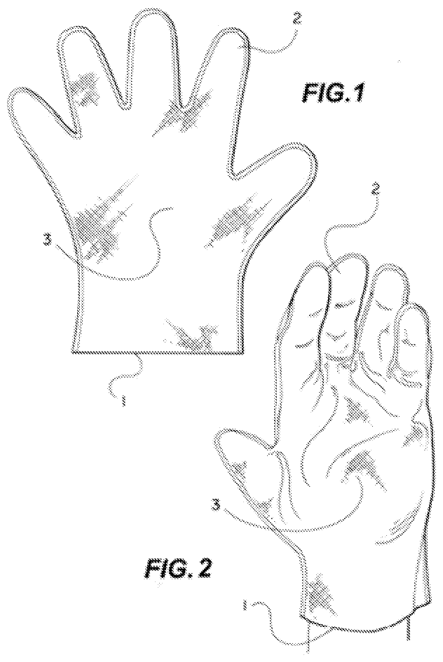

FIGS. 1 and 2 show embodiments of a nonwoven antibacterial hand glove. In the embodiment shown in FIGS. 1 and 2, the glove has an area to insert a wrist 1 and an area to insert a plurality of fingers 2 for ease of use of the glove. In both FIGS. 1 and 2, 3 is the of the glove that can be saturated with aqueous liquids or hydrophobic formulas to obtain different antibacterial uses of the gloves. FIGS. 1 and 2, 3 contains the contents of non-woven hydrophobic material and/or permeable material that is used to cover the human hand and protect with the aqueous sanitizes solution.

1. An antibacterial hand glove comprising:
a sheet that can be impregnated with an aqueous alcohol sanitizer solution, wherein the aqueous alcohol sanitizer comprises a hydrophobic functional ingredient entrapped in a plurality of polymeric beads, the hydrophobic functional ingredient being formulated and applied near an exterior surface of the sheet.

2. The antibacterial hand glove of claim 1, wherein the plurality of polymeric beads are located on a plurality of surfaces of the sheet.

3. The antibacterial hand glove of claim 1, wherein the plurality of polymeric beads are in the form of a polymeric content that includes a sanitizing solution.

4. The antibacterial hand glove of claim 1, wherein the plurality of polymeric beads are microcapsules.

5. The antibacterial hand glove of claim 1, wherein the hydrophobic functional ingredient is alcohol present in an amount between 60 to 80% by weight of the sheet.

6. A method of rendering a hand glove antibacterial comprising:
applying the hand glove over, a hand;
impregnating an exterior surface of a sheet of the hand glove with an aqueous sanitizing solution; and
waiting for a controlled release of at least one functional ingredient of the aqueous sanitizing solution before cleaning an object.

7. The method of claim 6, wherein the aqueous sanitizing solution is applied in sprayon dry form.

8. The method of claim 6, wherein the aqueous sanitizing solution contents are applied in the form of a spray on liquid or a sulking wet bath.

9. The method of claim 6, wherein the aqueous sanitizing solution is applied before the sheet is impregnated with the sanitizing solution.

10. (canceled)

11. The method of claim 6, wherein the plurality of polymeric beads are mixed with the aqueous sanitizing solution when applied to the sheet.

12. The antibacterial hand glove of claim 1, wherein the sheet is porous.

13. The antibacterial hand glove of claim 1, wherein the sheet is hydrophobic.

14. The antibacterial hand glove of claim 1, wherein the sheet is non-woven.

15. The method of claim 6, wherein the sheet is porous.

16. The method of claim 6, wherein the sheet is hydrophobic.

17. The method of claim 6, wherein the sheet is non-woven.

18. A cleaning kit comprising:
a hand glove comprising a non-woven porous sheet or a hydrophobic sheet; and
an aqueous solution, wherein the aqueous solution is applied to an exterior surface of the hand glove.

19. The cleaning kit of claim 18, wherein the aqueous solution is an alcohol based sanitizing solution.

20. The cleaning kit of claim 18, wherein the aqueous solution is a non-alcohol based sanitizing solution.

21. The cleaning kit of claim 18, wherein the aqueous solution is applied in at least one of the following ways: a sprayon dry form, a spray on liquid, or a sulking wet bath.
